(19) United States
(12) Patent Application Publication
(10) Pub. No.: US 2026/0144430 A1
(43) Pub. Date: May 28, 2026

(54) SPECULUM CAMERA ADAPTOR

(71) Applicant: ILLUMIGYN LTD., Neve Ilan (IL)

(72) Inventors: Gusti AVERBUCH, Modi'in (IL); Moshe SHTENGEL, Herzelia (IL); Orr ZION, Ness Ziona (IL); Itzhak LEVI, Nitzan (IL); Ronen FRIEDLANDER, Rohovot (IL)

(73) Assignee: ILLUMIGYN LTD., Neve Ilan (IL)

(21) Appl. No.: 19/120,271

(22) PCT Filed: Oct. 19, 2023
(86) PCT No.: PCT/IB2023/060575

§ 371 Date: Apr. 11, 2025

Related U.S. Application Data
(60) Provisional application No. 63/417,332, filed on Oct. 19, 2022.

(51) Int. Cl. A61B 1/00; A61B 1/05; A61B 1/227; A61B 1/233; A61B 1/303; A61B 1/31; A61B 1/32

(52) U.S. Cl. CPC A61B 1/00128; A61B 1/00103; A61B 1/00105; A61B 1/053; A61B 1/227; A61B 1/233; A61B 1/303; A61B 1/31; A61B 1/32

(57) ABSTRACT

Systems and devices for connecting a speculum device with different sizes of imaging devices without hindering the observable view are disclosed. An aspect of the device comprises an adaptor device comprising a back end configured to mate with an imaging device providing a substantially unhindered view of an area under analysis. The adaptor device also comprises a front end configured to connect to the back side of a speculum which does not block the line of sight. The adaptor device further comprises a opening for a lens of the imaging device to view a body part through the line of sight of the speculum.

### FIELD OF THE INVENTION

The disclosure herein relates to an optical speculum adaptor. In particular, a vaginal speculum adapter is described which can be used to connect a speculum with an imaging device.

### BACKGROUND OF THE INVENTION

A speculum is a duck-bill-shaped medical device for investigating body orifices. The doctors use it to see inside a hollow part of your body and diagnose or treat disease. One common use of the speculum is for vaginal exams. Gynecologists use it to open the walls of the vagina and examine the vagina and cervix.

A known speculum is the bivalved vaginal speculum having two blades that are hinged and are “closed” when the speculum is inserted to facilitate its entry and “opened” in its final position where they can be arrested by a screw mechanism, so that the operator is freed from keeping the blades apart. A doctor can examine the vagina using an imaging device which can be attached to the speculum. FIG. 1 illustrates a schematic view of a bivalved vaginal speculum 100 having hinged blades 101 and 102. An imaging device such as a still image or a video camera can connect to the speculum 100 opening from the back side 103. However, in this configuration, the inserted imaging device in the specula openings can substantially block the doctor's view of and access to an area of interest. This poses the risk of missing the detection of abnormal cells.

FIG. 2 illustrates a plastic single use speculum 200 which could attach to a camera and does not block the line of sight. The speculum device 200 is assembled on and mechanically screwed thereto on the imaging device which can capture images or video of cells within the target tissue. However, these disposable speculums are single use device and increases the cost of treatment. Further, since the speculum is assembled on the imaging device, these may not be adaptable for different size of imaging devices.

FIG. 3 illustrates a vaginal speculum of Gynex Corporation including an upper assembly 301 and a lower assembly 302. The upper assembly 301 includes a first spoon portion 303 and a single support bar 305 connecting the first spoon portion 303 to a first handle portion 306. The lower assembly 302 includes a second spoon portion 304 and a second handle portion 307. The single support bar of the upper assembly provides an unobstructed view of an area between the first spoon portion and the second spoon portion. The imaging device can connect to the back side of the speculum providing a substantially unhindered view of an area within a vagina under observation during a routine medical procedure. However, the opening of the speculum may not be adaptable for different size of imaging devices restricting its use for specific size of the imaging devices.

Thus, there is a need for a device or a system which can adapt to connect the speculum device with different size of imaging devices without hindering the observable view. The invention described herein addresses the above-described needs.

### SUMMARY OF THE EMBODIMENTS

In one aspect of the invention, an adaptor device for connecting a speculum device with different size of imaging devices without hindering the observable view is disclosed. The adaptor device comprises a front end configured to mate with the speculum device, wherein the front end comprises an elongated part which connects with a back side of the speculum device with a tight fit. The adaptor device also comprises a back end configured to mate with a front side of the imaging device, wherein the back end comprises a first opening for insertion of the front side of the imaging device comprising a viewing lens. The adaptor device further comprises a second opening for the lens of the imaging device to view a body part through the line of sight of the speculum.

As appropriate, the second opening is of smaller size than the first opening.

As appropriate, the back end comprises a covered part with the second opening of similar size as the first opening.

As appropriate, the second opening provides an unobstructed view of an area between a first spoon portion and a second spoon portion of the speculum device for the imaging device to view the body part.

As appropriate, the back end comprises a curved shaped part protruding outward enabling it to be connected to varied size of the imaging devices.

As appropriate, the adaptor device may be formed from a flexible material for enabling it to connect with different size and shapes of the imaging devices and the speculum devices.

As appropriate, the imaging device may be a digital still camera or a video camera configured to capture images in one or more file formats.

As appropriate, the speculum device may comprise one or more of a vaginal speculum device for examining the vagina and cervix, an anal speculum device for diagnosing diseases of the anus and rectum, an ear speculum device for examining the eardrum and ear canal and a nasal speculum device for examining the nose diseases.

As appropriate, the front end comprises an aperture which fits on the back side of the speculum device.

As appropriate, the aperture fits on an ‘L’ shaped part (506) of the speculum device.

As appropriate, the aperture may be of any shape including rectangular, circular, oval, triangular, hexagonal or any irregular shape.

### BRIEF DESCRIPTION OF THE FIGURES

For a better understanding of the embodiments and to show how it may be carried into effect, reference will now be made, purely by way of example, to the accompanying drawings.

With specific reference now to the drawings in detail, it is stressed that the particulars shown are by way of example and for purposes of illustrative discussion of selected embodiments only and are presented in the cause of providing what is believed to be the most useful and readily understood description of the principles and conceptual aspects. In this regard, no attempt is made to show structural details in more detail than is necessary for a fundamental understanding; the description taken with the drawings making apparent to those skilled in the art how the various selected embodiments may be put into practice. In the accompanying drawings:

FIG. 1 illustrates a schematic view of a bivalved vaginal speculum as known in the art;

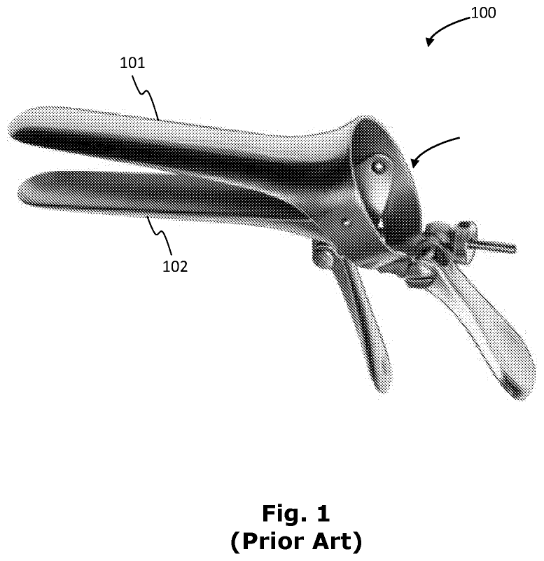

FIG. 2 illustrates a schematic view of a plastic single use speculum as known in the art;

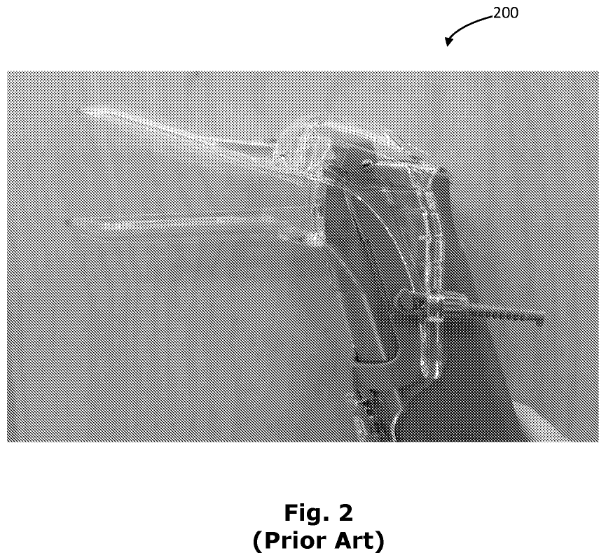

FIG. 3 illustrates a schematic view of a vaginal speculum of Gynex Corporation as known in the art;

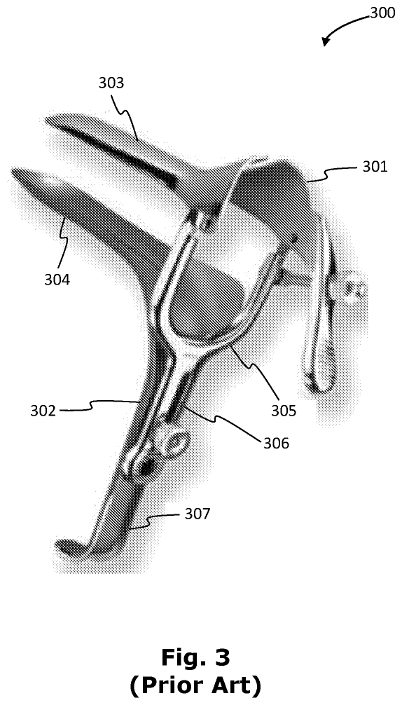

FIG. 4 illustrates a schematic view of a speculum adaptor configured to connect a speculum with an imaging device according to an aspect of the invention;

FIG. 5a illustrates a back end of the adaptor which connects with the imaging device according to an aspect of the invention;

FIG. 5b illustrates a back end of the adaptor with a covered part according to another aspect of the invention;

FIG. 6 illustrates a front end of the adaptor which can clip onto back of the speculum; and

FIG. 7 illustrates a schematic view of a combined system of the speculum and adaptor fitted with the imaging device.

### DESCRIPTION OF THE SELECTED EMBODIMENTS

Aspects of the present disclosure relate to a device or a system which can adapt to connect the speculum device with different size of imaging devices without hindering the observable view. In particular, the disclosure relates to an adaptor device configured to connect to the back side of a speculum which does not block the line of sight. The imaging device such as a camera connects to the back side of the adaptor providing a substantially unhindered view of an area within a vagina under observation during a routine medical procedure.

In particular examples of the system, the speculum device is a vaginal speculum configured to allow a physician to view and operate on areas within the vagina that would otherwise be inaccessible.

In other examples of the system, the adaptor device is configured to connect the speculum device with different size of imaging devices without hindering the observable view.

As required, the detailed embodiments of the present invention are disclosed herein; however, it is to be understood that the disclosed embodiments are merely examples of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

It is particularly noted that the systems and methods of the disclosure herein may not be limited in its application to the details of construction and the arrangement of the components or methods set forth in the description or illustrated in the drawings and examples. The systems and methods of the disclosure may be capable of other embodiments, or of being practiced and carried out in various ways and technologies.

Alternative methods and materials similar or equivalent to those described herein may be used in the practice or testing of embodiments of the disclosure. Nevertheless, particular methods and materials described herein for illustrative purposes only. The materials, methods, and examples not intended to be necessarily limiting. Accordingly, various embodiments may omit, substitute, or add various procedures or components as appropriate. For instance, the methods may be performed in an order different from described, and that various steps may be added, omitted or combined. In addition, aspects and components described with respect to certain embodiments may be combined in various other embodiments.

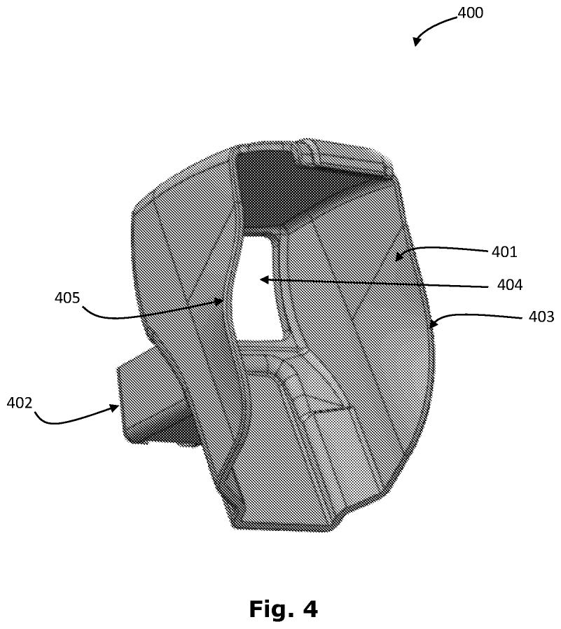

Reference is now made to FIG. 4, illustrates a schematic view of a speculum adaptor 400 configured to connect a speculum with an imaging device according to an aspect of the invention. The adaptor 400 comprises a back end 401 configured to mate with the imaging device and a front end 402 configured to mate with the vaginal speculum.

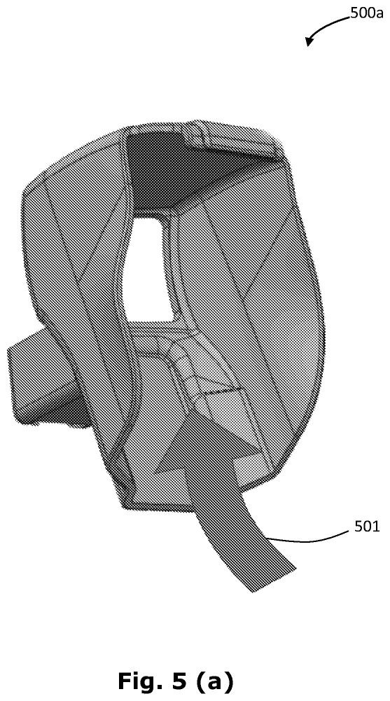

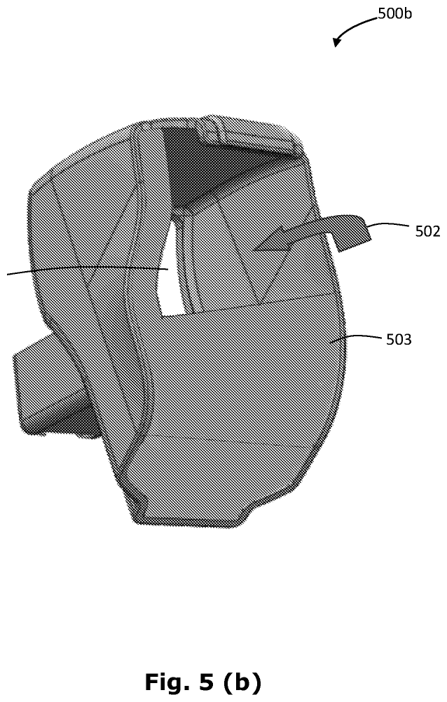

The back end 401 of the adaptor has a large opening 403. The imaging device is inserted into the adaptor 400 from the opening 403 and clicks into the adaptor 400 for a tight fit as illustrated in FIG. 5a. The arrow 501 shows the entry area of the imaging device. The adaptor 400 also comprises an opening 404 for the lens of the imaging device to view a vaginal part through the line of sight of the speculum. The opening 404 may be of smaller size than the opening 403. In an alternative embodiment, the back end has a covered part with opening 403 of same size and shape as of opening 404. As illustrated in FIG. 5b, the back end has a covered part 503 with the opening 502 similar in size as the opening 504. In such a configuration, an imaging device having long protruding lens fits within the openings 502 and 504.

In a particular embodiment, the back end 401 of the adaptor 400 has a curved shape 405 with protruded outward a little enabling it to be connected to varied size of imaging devices. The adaptor device may be formed from a flexible material for enabling it to fit with different size and shapes of the imaging device and the speculum. For example, the material may be PVC flexible plastic, fiberglass, Plexiglass, Polypropylene, thermoplastics, flexible metals, and others.

The imaging device may be a digital still camera which converts captured image into an electric signal upon a specific control or can be a video camera configured to capture images in one or more file formats. The video camera may be configured for any video file format including, but not limited to, Audio Video Interleave (AVI), Moving Picture Experts Group (MPEG)-4 (MP4), Apple QuickTime Movies (e.g., a MOV file), Microsoft WMV, Flash Video (FLV), Audio Video Interleave (AVI), Matroska Multimedia Container (MKV), WebM and so forth.

In addition to the vaginal speculum for examining the vagina and cervix, the speculum may also be an anal speculum for diagnosing diseases of the anus and rectum, an ear speculum for examining the eardrum and ear canal, a nasal speculum for examining the inside of the nose and more. The speculum may be used for a multitude of medical purposes without limiting the scope of the invention.

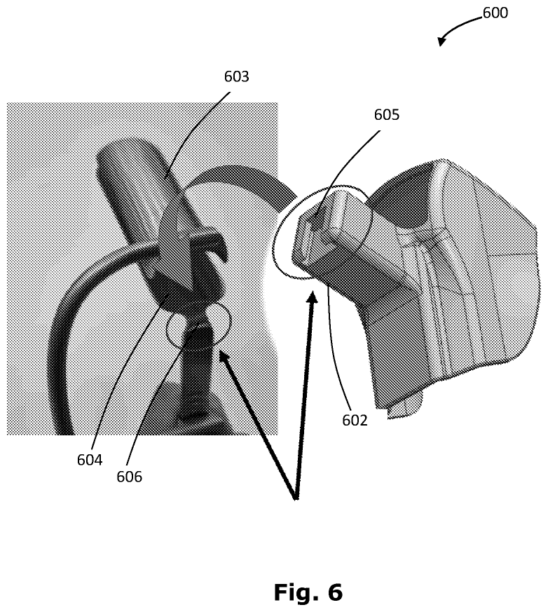

Reference is now made to FIG. 6 which illustrates a front end 602 of the adaptor 600 which can clip onto back of the speculum 603. The front end 602 of the adaptor has an elongated part which is inserted into the back 604 of the speculum 603 with a tight fit. The front end is shown to have a rectangular aperture 605 (shown in circle) which fits on the ‘L’ shaped part 606 of the speculum 603 (shown in circle). No further adjustment is required to align the adaptor 600 with the speculum 603 providing a direct line of sight for the imaging device. The size and shape of the front end 602 is selected to fit most of the standard speculums available in the market.

In a particular embodiment, the aperture 605 on the front end 602 is shown to be of rectangular shape which is to be fitted on the back of the speculum 603. Alternatively, the aperture 605 may be of different shape, such as, circular, triangular, hexagonal, etc. without limiting the scope of the invention. Further, the aperture 605 may be of an irregular shape.

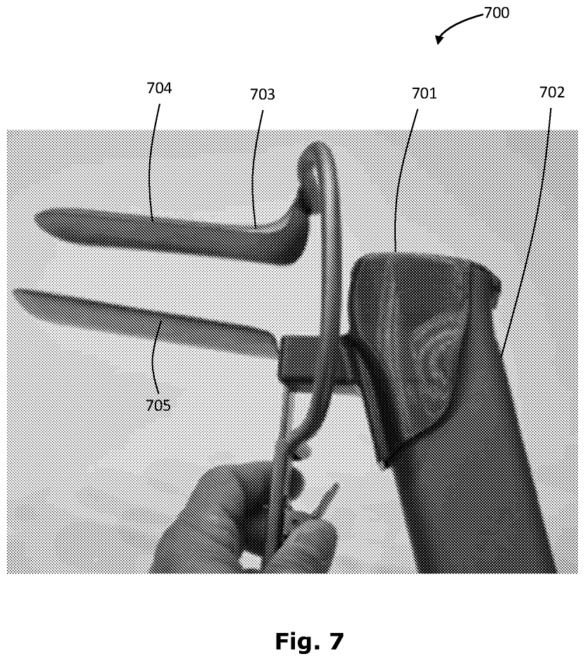

Reference is now made to FIG. 7 which illustrates a schematic view of a combined system 700 of the speculum 703 and adaptor 701 fitted with the imaging device 702. The adaptor 701 provides an unobstructed view of an area between a first spoon portion 704 and a second spoon portion 705 of the speculum 703 for the imaging device 702.

The systems and devices explained above may allow a speculum device to connect with an imaging device without hindering the observable view.

I/We claim

1. An adaptor device configured for connecting a speculum device with an imaging device, the adaptor device comprising:
a front end (402) configured to mate with the speculum device, wherein the front end comprises an elongated part which connects with a back side of the speculum device with a tight fit;
a back end (401) configured to mate with a front side of the imaging device, wherein the back end comprises a first opening (403) for insertion of the front side of the imaging device comprising a viewing lens; and
a second opening (404) for the lens of the imaging device to view a body part through the line of sight of the speculum.

2. The adaptor device of claim 1, wherein the second opening is of smaller size than the first opening.

3. The adaptor device of claim 1, wherein the back end comprises a covered part (503) with the second opening of similar size as the first opening.

4. The adaptor device of claim 1, wherein the second opening provides an unobstructed view of an area between a first spoon portion (704) and a second spoon portion (705) of the speculum device for the imaging device to view the body part.

5. The adaptor device of claim 1, wherein the back end comprises a curved shaped part protruding outward enabling it to be connected to varied size of the imaging devices.

6. The adaptor device of claim 1, wherein the adaptor device may be formed from a flexible material for enabling it to connect with different size and shapes of the imaging devices and the speculum devices.

7. The adaptor device of claim 6, wherein the flexible material comprises one or more of PVC flexible plastic, fiberglass, Plexiglass, Polypropylene, thermoplastics and flexible metals.

8. The adaptor device of claim 1, wherein the imaging device may be a digital still camera or a video camera configured to capture images in one or more file formats.

9. The adaptor device of claim 1, wherein the speculum device may comprise one or more of a vaginal speculum device for examining the vagina and cervix, an anal speculum device for diagnosing diseases of the anus and rectum, an ear speculum device for examining the eardrum and ear canal and a nasal speculum device for examining the nose diseases.

10. The adaptor device of claim 1, wherein the front end comprises an aperture (605) which fits on the back side of the speculum device.

11. The adaptor device of claim 10, wherein the aperture fits on an ‘L’ shaped part (606) of the speculum device.

12. The adaptor device of claim 10, wherein the aperture may be of any shape including rectangular, circular, oval, triangular, hexagonal or any irregular shape.
